(19) United States
(12) Patent Application Publication
(10) Pub. No.: US 2007/0283455 A1
(43) Pub. Date: Dec. 6, 2007

(54) Genetic Transformation of Grapevines

(76) Inventors: Dennis J. Gray, Howey In The Hills, FL (US); Manjul Dutt, Apopka, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/421,122

(22) Filed: May 31, 2006

(51) Int. Cl. A01H 5/00; C12N 15/82

(52) U.S. Cl. 800/278; 435/468

(57) ABSTRACT

Disclosed herein are methods of transforming grapevine. The methods involve the culturing of grapevine explant to induce shoot formation having meristematic regions and transforming meristematic tissue using Agrobacterium or particle bombardment.

### BACKGROUND

Plant genetic transformation is a process involving transfer of a desired gene or genes into the inheritable germline of crops plants. The genetic material carried by an individual cell is thus altered by incorporation of foreign (exogenous) DNA into its genome. Ability to utilize genetic transformation in grapevine is desirable in order to circumvent barriers to conventional genetic improvement. Grapevine is a perennial fruit crop with a long life cycle. It is also genetically self incompatible so that sexual crosses between related parents result in a high percentage (often 100%) of lethal offspring. In a conventional breeding program, the selection cycles leading to release of a cultivar take at least 10 to 15 years. These obstacles make incorporation of a single genetic trait, such as disease resistance via conventional breeding difficult to impossible. Thus, genetic transformation offers opportunities for the improvement of grapevine while allowing the continued use of traditional cultivars of considerable importance (Gray et al., 2005).

Currently, somatic embryogenic cells and somatic embryos are the most common explant for transformation of grapevine and the process of using them has been afforded patent protection (Gray et al., 2001, 2002). Somatic cells and embryos are derived through the process of somatic embryogenesis, which is the initiation of embryos from plant somatic tissues (i.e. clones of a single parent) that closely resemble their zygotic (i.e. sexually produced) counterparts (Ammirato, 1983). Somatic embryos of grapevine proliferate from single epidermal or sub epidermal cells and somatic cells proliferate from simple cell division of existing somatic embryos (i.e. direct somatic embryogenesis) (Gray, 1992, 1995; Gray et al., 2002). However, a major drawback to use of embryogenic cultures for transgenic plant regeneration is that their initiation, proliferation and ability to undergo genetic transformation is cultivar dependent (i.e. only certain grapevine cultivars are responsive). Also, among responsive cultivars, embryogenic cultures variously are difficult to impossible to maintain over time, reducing their utility for use in genetic transformation. These drawbacks are illustrated by the relatively low number of grapevine species, varieties and hybrids for which somatic embryogenesis and transformation has been reported (Gray et al., 2005)

An alternative to use of embryogenic cultures for transformation is use of micropropagation cultures. Micropropagation cultures are easier to initiate from a wide range of grapevine cultivars and can be maintained over time without loss of function. Micropropagation is the clonal propagation of plants using in vitroculture techniques where initial explants are vegetative shoot apices or axillary bud meristems. New shoots arise from these complex tissues (i.e. the process of somatic embryogenesis is not involved). In essence, apical or axillary bud meristem culture produces a miniaturized plant by shortening internodal length, resulting in compact shoots with multiple branches. This change in growth morphology is caused by cytokinin like plant growth regulators such as 6-benzylaminopurine (BAP) and kinetin, which inhibit root development and cause reduction of internodes, resulting in a large number of nodes per unit area. This is because cytokinins overcome apical dominance of axillary buds (Torregrossa et al., 2001). Thus cultures no longer display a typical vine morphology, but appear as dense proliferative masses of small shoots (Goussard,1982) as illustrated in FIG. 1. In vitro grapevine propagation on a large scale has been obtained by micropropagation of shoot meristem cultures (Chee and Pool, 1983; Gray and Fisher, 1985). Also production of pathogen free stock is a major application of micropropagation and was successfully used for virus elimination as early as 1964 (Galzy, 1964). The ease of maintaining micropropagation cultures coupled with a large number of genotypes that can be established in vitromakes them attractive target tissue for genetic transformation. Transformation techniques using micropropagated cultures might break the genotype specificity barrier that exists with somatic embryogenic cultures. This invention disclosure describes the production of transgenic grapevines using micropropated cultures. Stable genetically transformed plants were produced using this technique.

### BRIEF DESCRIPTION OF DRAWINGS

FIG. 1. Proliferative masses of in vitrograpevines.

FIG. 2. Diagram of the shoot tip nicking process.

FIG. 3. Transient expression after 3 days of co-cultivation with Agrobacterium at 260C. EGFP expression was visualized using a stereomicroscope equipped for epi-fluorescence Illumination.

FIG. 4. Stable transgenic grapevines. a. Transgene expression on stable shoots as visualized using a stereomicroscope equipped for epi-fluorescence illumination. b. Shoot tips as visualized using normal incandescent light. c. Transgenic rooted plants

FIG. 5. Stable transgene incorporation in eight selected lines as confirmed by PCR using EGFP specific primers.

FIG. 6. Diagram of the particle bombardment device.

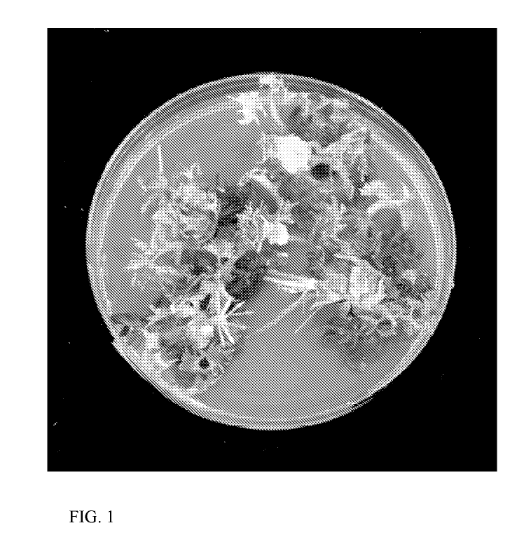

### DETAILED DESCRIPTION

The present invention discloses a method for the efficient transformation of grapevine plants involving introduction of an exogenous nucleic acid sequence. The exogenous nucleic acid sequence is preferably in the form of a plant transformation vector. Any vector suitable for the transformation of plants can be used in the present invention. A suitable plant transformation plasmid or vector typically contains a selectable or screenable marker and associated regulatory elements as described, along with one or more nucleic acids capable of being expressed in a plant in a manner sufficient to confer a particular desirable trait or phenotype to the plant. Examples of suitable structural genes of interest envisioned by the present invention would include but are not limited to genes for insect or pest (bacterial, fungal, nematocidal) tolerance; herbicide tolerance; genes for quality improvements such as yield, nutritional enhancements, environmental or stress tolerances; or any desirable changes in plant physiology, growth, development, morphology, or plant product(s).

Alternatively, the DNA coding sequences can affect these phenotypes by encoding a non-translatable RNA molecule that causes the targeted inhibition of expression of an endogenous gene, for example via antisense- or cosuppression-mediated mechanisms (see, for example, Bird et al., Biotech. Gen. Engin. Rev. 9:207, 1991). The RNA could also be a catalytic RNA molecule (i.e., a ribozyme) engineered to cleave a desired endogenous mRNA product (see for example, Gibson and Shillitoe, Mol. Biotech. 7:125, 1997). Thus, any gene that produces a protein or mRNA that expresses a phenotype or morphology change of interest is useful for the practice of the present invention.

Exemplary nucleic acids that may be introduced by the methods encompassed by the present invention include for example, DNA sequences or genes from another species, or genes or sequences that originate with or are present in the same species, but are incorporated into recipient cells by genetic engineering methods rather than classical reproduction or breeding techniques. However, the term exogenous is also intended to refer to genes that are not normally present in the cell being transformed; or perhaps simply not present in the form, structure, etc., as found in the transforming DNA segment or gene; or genes that are normally present yet that one desires, e.g., to have over-expressed. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

Plant transformation vectors generally contain one or more nucleic acid coding sequences of interest under the transcriptional control of 5′ and 3′ regulatory sequences. Such vectors generally comprise, operatively linked in sequence in the 5′ to 3′ direction, a promoter sequence that directs the transcription of a downstream structural nucleic acid sequence in a plant; optionally, a 5′ non-translated leader sequence; a nucleic acid sequence that encodes a protein of interest; and a 3′ non-translated region that encodes a polyadenylation signal that functions in plant cells to cause the termination of transcription and the addition of polyadenylate nucleotides to the 3′ end of the mRNA encoding the protein. The promoter may be homologous or heterologous to the structural nucleic acid sequence. Typical 5′ to 3′ regulatory sequences include a transcription initiation start site, a ribosome binding site, an RNA processing signal, a transcription termination site, and/or a polyadenylation signal. Plant transformation vectors also generally contain a selectable or screenable marker. Expression of the selectable or screenable marker sequence results in a phenotype that allows the differentiation of transgenic and non-transgenic grapevine tissues.

Any selectable marker suitable for plant transformation may be used in the present invention. Examples of selectable markers reported to be functional in plants include EPSPS (Klee et al., Mol. Gen. Genet. 210(3): 437, 1987), neomycin phosphotransferase (Loopstra et al., Plant Mol. Biol. 15(1): 1, 1990), hygromycin phosphotransferase (Meijer et al., Plant Mol. Biol. 16(5): 807, 1991), resistance to methotrexate (Irdani et al., Plant Mol. Biol. 37(6): 1079, 1998), phosphinothricin acetyl transferase (Shen et al., J. Gen. Virol. 76: 965, 1995), and chlorsulfuron resistance (Lee et al., Plant Cell 2(5): 415, 1990). Screenable markers reported to be functional in plants include GFP (Niwa et al., Plant J. 18(4): 455, 1999), GUS (Suzuki et al., Plant Cell. Physiol. 40(3): 289, 1999), CAT (Leisy et al., Plant Mol. Biol. 14(1): 41, 1990), and luciferase (Macknight et al., Plant Mol. Biol. 27(3): 457, 1995). In some cases, the structural nucleic acid coding sequence can also function as the selectable or screenable marker sequence.

In light of this disclosure, numerous other possible selectable or screenable marker genes, regulatory elements, and other sequences of interest will be apparent to those of skill in the art. Therefore, the foregoing discussion is intended to be exemplary rather than exhaustive.

Plant explant material obtained from grapevine can be cultured such that multiple shoots are generated. In one embodiment, shoot tips obtained from grapevine are excised and cultured (i.e., plated) on a culture medium designed to encourage shoot formation (SF culture medium). Typically, the shoot tip comprises both apical and axillary meristem regions. Of course, other plant tissue may be used in the present invention to produce multiple shoot cultures. SF medium typically comprises MS salts, a carbohydrate source (preferably sucrose), B₅ vitamins and a gelling agent such as PHYTAGEL.™. In addition, the SF culture medium typically contains at least one cytokinin-like growth regulator such as BA, kinetin, 2ip, zeatin and the like. However, the requirement for a cytokinin-like growth regulator may be reduced or eliminated by changing environmental growth conditions of the cultures, such as temperature, light level and duration, such that the plant tissue grows in a manner similar to tissue grown with added cytokinin. BA is the preferred growth regulator for inducing shoot meristematic cultures from apical or axillary meristems. The cytokinin-like growth regulator may be present in the SF culture medium at a concentration as low as about 2.0 mg/L, or about 1.0 mg/L, or about 0.05 mg/L or about 0.01 mg/L, or even lower; alternatively, the cytokinin-like growth regulators may be present in the SF culture medium at a concentration as high as about 5 mg/L, or about 8 mg/L, or 10 mg/L, or about 25, or about 100 mg/L, or even higher. In one embodiment, the cytokinin-like growth regulator is present in the SF culture medium in from about 0.05 mg/L to about 25 mg/L, more preferably from 0.1 mg/L to 10 mg/L, and most preferably from about 0.5 mg/L to about 8 mg/L. Additional growth regulators may be added to the SF culture medium to induce shoot meristematic cultures. Such growth regulators include (gibberellic acid (GA), and those with auxin-like function such as indole-3-acetic acid (IAA), .alpha.-naphthaleneacetic acid (NAA), thidiazuron (TDZ), 3,6-dichloro-o-anisic acid (DICAMBA), 2,4,5,-trichlorophenoxyacetic acid (2,4,5-T), 2,4-dichlorophenoxyacetic acid(2,4-D), and other growth regulators known to those skilled in the art.

In the case of transforming grapevine cells with Agrobacteria, those skilled in the art will appreciate, in view of the teachings herein that, any suitable Agrobacterium vector or vector system for transforming the plant may be employed. A variety of Agrobacterium strains are known in the art and may be used in the methods of the invention. Representative Agrobacterium vector systems are described in G. An, et al. EMBO J. 4, 277 (1985); L. Herrera-Estrella, et al., Nature 303, 209 (1983); L. Herrera-Estrella et al., EMBO J. 2, 987 (1983); L. Herrera-Estrella et al., in Plant Genetic Engineering (Cambridge University Press, New York, page 63 (1985); Hooykaas, Plant Mol. Biol. 13, 327 (1989); Smith et al., Crop Science 35, 301 (1995); Chilton, Proc. Natl. Acad. Sci. USA 90, 3119 (1993); Mollony et al., Monograph Theor. Appl. Genet. NY 19, 148 (1993); Ishida et al., Nature Biotechnol. 14, 745 (1996); and Komari et al., The Plant Journal 10, 165 (1996), the disclosures of which are incorporated herein by reference in their entirety.

In addition to the T-region of Agrobacterium, the Ti (or Ri) plasmid contains a vir region. The vir region is important for efficient transformation, and may be species-specific. Binary vector systems have been developed where the manipulated disarmed T-DNA carrying, for example, heterologous DNA and the vir functions are present on separate plasmids. In other words, a heterologous nucleic acid sequence (i.e., gene or genes) of interest and the flanking T-DNA can be carried by a binary vector lacking the vir region. The vir region is then provided on a disarmed Ti-plasmid or on a second binary plasmid. In this manner, a modified T-DNA region comprising heterologous DNA is constructed in a small plasmid which replicates in E coli. This plasmid is transferred conjugatively in a tri-parental mating or via electroporation into A. tumefaciens that contains a compatible plasmid with virulence gene sequences. The vir functions are supplied in trans to transfer the T-DNA into the plant genome. As another alternative, the heterologous nucleic acid sequence and the T-DNA border sequences can be put into the T-DNA site on the Ti-plasmid through a double recombination event by which the new T-DNA replaces the original Ti-plasmid T-DNA. The vir region can be supplied by the Ti-plasmid or on a binary plasmid. As yet a further alternative, the heterologous nucleic acid sequence and flanking T-DNA can be integrated into the bacterial chromosome as described by U.S. Pat. No. 4,940,838 to Schilperoort et al., and the vir region can then be supplied on a Ti-plasmid or on a binary plasmid. Binary vectors as described herein may be used in the practice of the present invention, and are preferred.

Alternatively, in other embodiments of the invention, super-binary or “supervirulent” Agrobacterium vectors are employed in the Agrobacterium solutions. See, e.g., U.S. Pat. No. 5,591,615 and EP 0 604 662, herein incorporated by reference. Such a super-binary vector has been constructed containing a DNA region originating from the hypervirulence region of the Ti plasmid pTiBo542 (Jin et al., J. Bacteriol. 169, 4417 (1987)) contained in a super-virulent A. tumefaciens A281 exhibiting extremely high transformation efficiency (Hood et al., Biotechnol. 2, 702 (1984); Hood et al., J. Bacteriol. 168, 1283 (1986); Komari et al., J. Bacteriol. 166, 88 (1986); Jin et al., J. Bacteriol. 169, 4417 (1987); Komari, Plant Science 60, 223 (1987); ATCC Accession No. 37394.

Exemplary super-binary vectors known to those skilled in the art include pTOK162 (see Japanese Patent Appl. (Kokai) No. 4-222527, European Patent Applications EP 504,869 and EP 604,662, and U.S. Pat. No. 5,591,616, herein incorporated by reference) and pTOK233 (see Komari, Plant Cell Reports 9,303 (1990), and Ishida et al., Nature Biotechnology 14, 745 (1996); herein incorporated by reference). Other super-binary vectors may be constructed by the methods set forth in the above references. Super-binary vector pTOK162 is capable of replication in both E. coli and in A. tumefaciens. Additionally, the vector contains the virB, virC and virG genes from the virulence region of pTiBo542. The plasmid also contains an antibiotic resistance gene, a selectable marker gene, and, if desired, a nucleic acid of interest to be transformed into the plant. Super-binary vectors of the invention can be constructed having the features described above for pTOK162. The T-region of the super-binary vectors and other vectors for use in the invention may be constructed to have restriction sites for the insertion of, for example, heterologous genes to be delivered to the plant. Alternatively, heterologous nucleic acids to be transformed can be inserted in the T-DNA region of the vector by utilizing in vivo homologous recombination. See, Herrera-Esterella et al., EMBO J. 2, 987 (1983); Horch et al., Science 223, 496 (1984). Such homologous recombination relies on the fact that the super-binary vector has a region homologous with a region of pBR322 or other similar plasmids. Thus, when the two plasmids are brought together, a desired gene is inserted into the super-binary vector by genetic recombination via the homologous regions.

In exemplary embodiments, Agrobacterium vectors and vector systems utilized in the methods of the present invention are modified by recombinant nucleic acid techniques to contain a heterologous nucleic acid (e.g., a gene or genes of interest) to be expressed in the transformed cells. “Expression” refers to the transcription and translation of a structural heterologous nucleic acid to yield the encoded protein. Expression may also refer to transcription only, as for example in the case of antisense constructs. The heterologous nucleic acid to be expressed is preferably incorporated into the T-region and is flanked by T-DNA border sequences of the Agrobacterium vector.

The methods of the invention are generally applicable for a variety of grape plants (for example, Vitis spp., Vitis spp. hybrids, and all members of the subgenera Euvitis and Muscadinia), including scion or rootstock cultivars. Exemplary scion cultivars include, without limitation, those which are referred to as table or raisin grapes Alden, Almeria, Anab-E-Shahi, Autumn Black, Beauty Seedless, Black Corinth, Black Damascus, Black Malvoisie, Black Prince, Blackrose, Bronx Seedless, Burgrave, Calmeria, Campbell Early, Canner, Cardinal, Catawba, Christmas, Concord, Dattier, Delight, Diamond, Dizmar, Duchess, Early Muscat, Emerald Seedless, Emperor, Exotic, Ferdinand de Lesseps, Fiesta, Flame seedless, Flame Tokay, Gasconade, Gold, Himrod, Hunisa, Hussiene, Isabella, Italia, July Muscat, Khandahar, Katta, Kourgane, Kishmishi, Loose Perlette, Malaga, Monukka, Muscat of Alexandria, Muscat Flame, Muscat Hamburg, New York Muscat, Niabell, Niagara, Olivette blanche, Ontario, Pierce, Queen, Red Malaga, Ribier, Rish Baba, Romulus, Ruby Seedless, Schuyler, Seneca, Suavis (IP 365), Thompson seedless, and Thomuscat. They also include those used in wine production, such as Aleatico, Alicante Bouschet, Aligote, Alvarelhao, Aramon, Baco blanc (22A), Burger, Cabernet franc, Cabernet, Sauvignon, Calzin, Carignane, Charbono, Chardonnay (e.g., CH 01, CH 02, CH Dijon), Chasselas dore, Chenin blanc, Clairette blanche, Early Burgundy, Emerald Riesling, Feher Szagos, Fernao Pires, Flora, French Colombard, Fresia, Furmint, Gamay, Gewurztraminer, Grand noir, Gray Riesling, Green Hungarian, Green Veltliner, Grenache, Grillo, Helena, Inzolia, Lagrein, Lambrusco de Salamino, Malbec, Malvasia bianca, Mataro, Melon, Merlot, Meunier, Mission, Montua de Pilas, Muscadelle du Bordelais, Muscat blanc, Muscat Ottonel, Muscat Saint-Vallier, Nebbiolo, Nebbiolo fino, Nebbiolo Lampia, Orange Muscat, Palomino, Pedro Ximenes, Petit Bouschet, Petite Sirah, Peverella, Pinot noir, Pinot Saint-George, Primitivo di Gioa, Red Veltliner, Refosco, Rkatsiteli, Royalty, Rubired, Ruby Cabernet, Saint-Emilion, Saint Macaire, Salvador, Sangiovese, Sauvignon blanc, Sauvignon gris, Sauvignon vert, Scarlet, Seibel 5279, Seibel 9110, Seibel 13053, Semillon, Servant, Shiraz, Souzao, Sultana Crimson, Sylvaner, Tannat, Teroldico, Tinta Madeira, Tinto cao, Touriga, Traminer, Trebbiano Toscano, Trousseau, Valdepenas, Viognier, Walschriesling, White Riesling, and Zinfandel. Rootstock cultivars include Couderc 1202, Couderc 1613, Couderc 1616, Couderc 3309 (Vitis riparia X rupestris), Dog Ridge, Foex 33 EM, Freedom, Ganzin 1 (A.times.R #1), Harmony, Kober 5BB, LN33, Millardet & de Grasset 41B (Vitis vinifera X berlandieri), Millardet & de Grasset 420A, Millardet & de Grasset 101-14 (Vitis riparia X rupestris), Oppenheim 4 (SO.sub.4), Paulsen 775, Paulsen 1045, Paulsen 1103, Richter 99, Richter 110, Riparia Gloire, Ruggeri 225, Saint-George, Salt Creek, Teleki 5A, Vitis rupestris Constantia, Vitis california, and Vitis girdiana, Vitis rotundifolia, Vitis rotundifolia Carlos, Teleki 5C (Vitis berlandieriX riparia), 5BB Teleki (selection Kober, Vitis berlandieri X riparia), SO.sub.4 (Vitis berlandieriX rupestris), and 039-16 (Vitis vinifera X Muscadinia).

### EXAMPLES

1. Establishment of In Vitro Cultures

In vitrocultures were established on C2D medium (Chee et al., 1984) containing 4 μM of 6-benzylaminopurine (henceforth called C2D4B) and solidified with 7.0 g 1⁻¹ TC agar (Phytotechnology laboratories LLC, Shawnee Mission, KS, USA) using the method previously described by Gray and Benton, (1991). The cultures were maintained at 25° C. with a 16 hour light/8 hour dark cycle using cool white fluorescent lights. Shoot tips were harvested from the stock cultures and moved into fresh C2D4B to maintain the stock cultures. To bulk up cultures for the experiments, both nodes and shoot tips were taken from the stock cultures. After a week of incubation under lights, cultures were etiolated by placing them in a box so as to provide complete darkness and maintained at 25° C. Developing shoots were used after a month of culture for transformation studies.

2. Methods for Genetic Transformation of Micropropagated Cultures

Various wounding methods were studied in order to facilitate Agrobacterium infection of the apical meristem cells. Preliminary studies that did not incorporate a wounding technique did not result in transgenic plants.

a) Method Incorporating Nicking of Shoot Tips and nodes

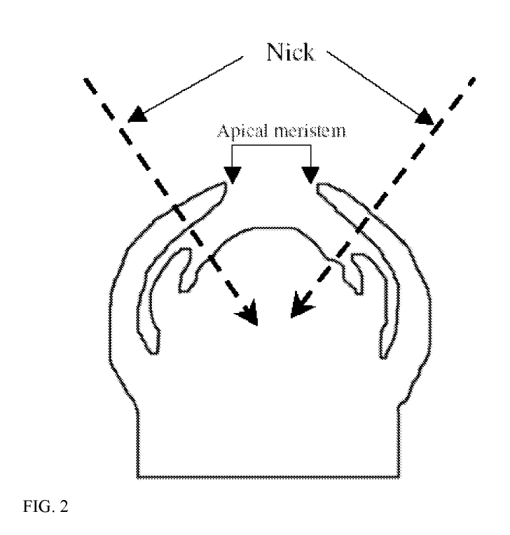

Agrobacterium tumefaciens strain EHA 105 containing a binary vector with a fusion selection-reporter gene (NPT II and EGFP) and gene of interest (e.g. a lytic peptide (Cecropin A) or other peptide fusion gene) under control of a bi-directional constitutive promoter (Li and Gray, 2002a, 2002b, Li et al., 2004) was used. Shoots and nodes were harvested and all surrounding appendages were removed in order to isolate the apex. The shoots and nodes were blotted dry on a filter paper and nicked using a sterile no. 11 stainless steel surgical blade (Feather Safety Razor Co Ltd., Japan). The nicks were made on or close to the apical dome (FIG. 2). 100 nicked shoots and nodes were placed in a 20 ml flask containing 0.5 g of sterile 320 grit carborundum (Fisher Scientific, Pittsburg, Pa, U.S.A) and 2 ml of the Agrobacterium culture with an OD value adjusted to 0.8. The 20 ml flask containing the in vitrocultures was placed in a Solid State Ultrasonic FS-14 (Fisher Scientific, Pittsburg, Pa, U.S.A) sonicator for 60 seconds. A further 3 ml of Agrobacterium culture was added for co-culture for an additional 9 minutes. Other treatments included nicked shoot tips and nodes directly co-cultured with Agrobacterium , shoot tips and nodes sonicated without carborendum and co-cultured with Agrobacterium or tips and nodes co-cultured in Agrobacterium cultures containing carborendum. The cultures were then blotted dry on a P8 7 cm diameter filter paper (Fisher Scientific, Pittsburg, Pa, U.S.A) and placed for co-cultivation in a petridish containing C2D4B soaked filter paper. Shoots were co-cultivated for 3 days at 26° C. in the dark. Unless otherwise noted, all operations were carried out in a sterile laminar flow hood.

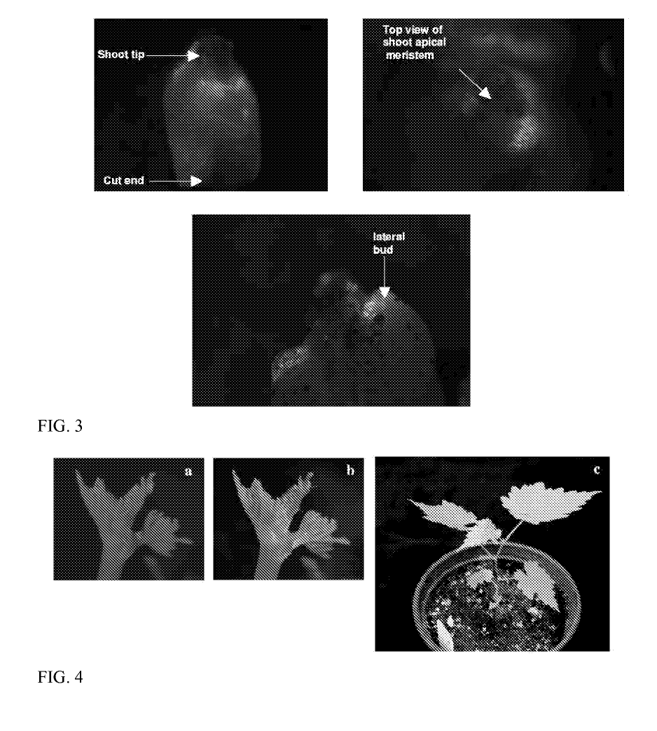

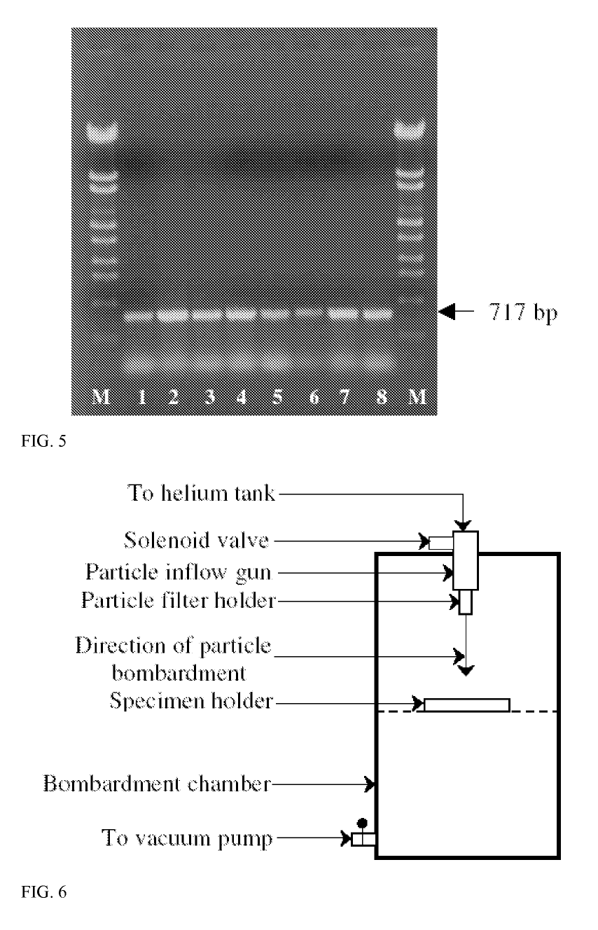

Transient Green Fluorescent Protein (GFP) expression, indicative of functional gene expression, but not necessarily stable integration of T-DNA into the plants DNA, was observed at the end of the co-cultivation period using a stereomicroscope equipped for epi-flourescence illumination (FIG. 3). The cultures with transient GFP expression were incubated in the dark for one day in 50 ml liquid C2D4Bcc (containing 200 mg 1⁻¹ each of carbenicillin and cefotaxime) medium on an orbital shaker at 120 rpm. Cultures were then placed on solidified C2D4B medium containing 200 mg 1⁻¹ each of carbenicillin and cefotaxime and 20 mg 1⁻¹ of kanamycin and subcultured every 2 weeks until stable transgenic plants were obtained as shown in FIG. 4. Stable transgene incorporation was confirmed by PCR (FIG. 5) in addition to GFP emission from all plant parts. Table 1 shows percentage of transient vs. stable shoot development.

b) Method Incorporating Fragmenting of the Shoot Tips

As an alternative to the use of nicking, shoot tips were harvested and the tips fragmented as per protocol outlined by Barlass and Skene (1978). Shoot tips, measuring 5 mm in length and containing 2-3 leaf primordia were harvested from in vitrogrowing plants. Individual tips were cut with a sterile no. 11 stainless steel surgical blade (Feather Safety Razor Co Ltd., Japan) into several fragments in a disposable polystyrene 100 ×15 mm petridish (Fisher Scientific, Pittsburg, Pa, U.S.A). Fragments were immediately placed into 500 μl of Agrobacterium culture containing the construct as described above and co-cultured for 10 minutes. The fragmented apices were further teased apart in co-culture medium. The fragmented apices were then blotted dry on a P8 7 cm diameter filter paper (Fisher Scientific, Pittsburg, Pa, U.S.A) and placed for co-cultivation in a petridish containing C2D4B soaked filter paper for 3 days at 26° C. in the dark. At the end of the co-cultivation period, cultures were moved into 50 ml Erlenmeyer flasks containing 10 ml of liquid C2D4Bcc medium in a shaker at 120 rpm overnight. The cultures were then moved into 10 ml of liquid C2D4Bcc and containing 20 mg 1⁻¹ of kanamycin for 2 weeks before moving them into solid medium as outlined above. Stable transgene incorporation was confirmed by PCR (FIG. 5).

c) Method Incorporating Biolistics (Particle Bombardment) of Shoot Tips

As an alternative to nicking or fragmenting, shoots and nodes were harvested as before and all surrounding appendages were removed in order to isolate the apex. The shoots and nodes were dried under a laminar flow hood for 10 minutes before being used for particle bombardment. M25 Tungsten (Bio-Rad Laboratories, Inc., Hercules, Ca.) having a median particle size of 1.7 μm was sterilized by placing 10 mg tungsten in 100 ml of 100% ethanol overnight. After 3 rinses with sterile water, the tungsten was resuspended in 25 μl sterile water for use in bombardment. The bombardment device used was as developed and described by Gray et al. (1994) (FIG. 6). For bombardment, a 5 μl drop of particle mixture was placed in the middle of a plastic filter holder screen and screwed tightly into the bombardment chamber. 10 shoot tips and nodes were placed in specimen holders, which were then placed in the bombardment chamber. Care was taken to orient apical meristems in the direction of plastic filter holder. The chamber was evacuated to 90 OkPa (27 in. Hg) vacuum using a vacuum pump. The timer was set at 0.1sec for firing the device to propel particles into the tissue, thus accomplishing wounding of shoot tips and nodes. The chamber was vented after firing and the specimens were replaced. All activities were carried out in a sterile laminar flow hood. Following wounding by bombardment with the tungsten particles, 100 shoot tips and nodes were placed in 5 ml Agrobacterium culture. Subsequent co-culture time, co-cultivation period, transient and stable plant selection was carried out as described earlier.

TABLE 1
Agrobacterium mediated transformation of
Vitis vinifera “Thompson Seedless”.

| Treatment | Transient Expression | Stable Lines |
|---|---|---|
| Nicking | 30 | 3 |
| Carborendum | 16 | 0 |
| Sonication | 4 | 0 |
| Nicking + Carborendum + | 86 | 13 |
| Sonication | | |
| Fragmenting | 14 | 2 |

### RELATED REFERENCES

Ammirato, P. V. (1983) Embryogenesis. In: Techniques for propagation and breeding. Evans, D. A., Sharp, W. R., Ammirato, P. V and Yamada, Y. (eds) Handbook of plant cell cultures, Vol. I, pp. 82-123.
Barlass, M. and Skene, K.G.M. (1978) In vitro propagation of grapevine (Vitis vinifera L. from fragmented shoot apices. Vitis 17:335-340.
Chee, R. and Pool, R. M. (1983) In vitro vegetative propagation of Vitis: application of previously defined culture conditions to a selection of genotypes. Vitis 22: 363-374.
Chee, R., Pool, R. M. and Bucher, D. (1984) A method for large scale in vitropropagation of Vitis. New York Food Life Sci. Bul. 109: 1-9.
Galzy, R. (1964) Technique de la thèrmotherapy des viruses de la vigne. Ann. Epiphyt. 15:245 -256.
Gray, D. J. (1992) Somatic embryogenesis and plant regeneration from immature zygotic embryos of muscadine grape (Vitis rotundifolia) cultivars. Am. J. Bot. 79: 542-546.
Gray, D. J. (1995) Grape. In: Jain, S. M., Gupta, P. K. and Newton, R. J. (eds) Somatic embryogenesis in woody plants, Vol. 2 Angiosperms. Kluwer Academic Publishers, Dordrecht, pp. 191-217.
Gray, D. J. and Fisher, L. C. (1985) In vitro shoot propagation of grape species, hybrids and cultivars. Proc. Fla. State Hort. Soc. 98: 172-174.
Gray, D.J. and Benton, C.M. (1991) In vitro micropropagation and plant establishment of muscadine grape cultivars (Vitis rotundifolia). Plant Cell Tissue Organ Culture 27: 7-14.
Gray, D. J., Hiebert, E., Lin C. M., Compton, M. E., McColley D. W., Harrison R. J., and Gaba, V. (1994). Simplified construction and performance of a device for particle bombardment. Plant Cell, Tissue and Organ Culture 37: 179-184.
Gray, D. J., Jayasankar, S., Li, Z., Cordts, J., Scorza, R. and Srinivasan, C. (2002) Transgenic grapevines. In: Khachatourians, G. G., McHughen, A., Scorza, R., Nip, W. and Hui, Y. H. (eds.) Transgenic plants and crops. New York: Marcel Dekker pp. 397-405.
Gray, D. J., Jayasankar, S. and Li, Z. T. (2005) Vitis spp. Grape. In: Biotechnology of Fruit and Nut Crops (ed. R. E. Litz) pp.672-706.
Goussard, P. G. (1982) Morphological responses of shoot apices of grapevine cultured in vitro: Effects of cytokinin in routine subculturing. Vitis 21: 293-298.
Huang, X. S. and Mullins, M. G. (1989) Application of biotechnology to transferring alien genes to grapevine. Heriditas (Beijing) 11: 9-11.
Levenko, B. A. and Rubtsova, M. A. (2000) Herbicide resistant transgenic plants of grapevine. Acta Hort. 528: 337 -339.
Li, Z. T., Jayasankar, S., and Gray, D. J. (2004). Bi-directional duplex promoters with duplicated enhancers significantly increase transgene expression in grape and tobacco.
Transgenic Research 13(2): 143-154.
Mezzetti, B., Pandolfini, T., Navacchi ,O., and Landi, L. (2002) Genetic transformation of Vitis vinifera via organogenesis. BMC Biotechnology 2:18-27.
Mullins, M. G., Tang, F. C., and Facciotti, D. (1990). Agrobacterium mediated genetic transformation of grapevines: Transgenic plants of Vitis rupestris Scheele and buds of Vitus vinifera. Bio/Technology 8:1041-1045.
Torregrosa, L., Bouquet, A. and Goussard P. G. (2001) In vitro culture and propagation of grapevine. In: K.A.Roubelakis-Angelakis (ed.) Molecular biology and biotechnology of the Grapevine pp. 281-326.
Gray, D. J., Subramanian, J. and Litz R E. (2001) Regeneration system for grape and uses thereof. South African Pat. No. 2000/0591.
Gray, D. J., Subramanian, J. and Litz R E. (2002) Regeneration system for grape and uses thereof. U.S. Pat. No. 6,455,312.
Fillatti, J. and Comai, L. (1989) Transformation and foreign gene expression with woody species. U.S. Pat. No. 4,795,855.
Kawazu, T., Doi, K., Kondo, K. (2003 )Process for transformation of mature trees of Eucalyptus plants. U.S. Pat. No. 6,563,024.
Li, Z. and Gray D. J. (2002a) Bi-directional dual promoter complex with enhanced promoter activity for transgene expression in eukaryotes. U.S. Pat. Application No.10/075,105.
Li, Z. and Gray D. J. (2002b) Bi-directional dual promoter complex with enhanced promoter activity for transgene expression in eukaryotes. International Application PCT/US02/0418.
Martinell, B., Julson, L. A., Hinchee, M. A. W., Connor-Ward, D., McCabe, D. and Emler, C. (1999) Efficiency soybean transformation protocol. U.S. Pat. No. 5,914,451
Smith, R. H., Gould, J. H. and Ulian, E. (1992) Method for transforming plants via the shoot apex. U.S.Pat. No. 5,164,310.
Young, M. M. and Reichert, N. A. (2003) Methods for maize transformation coupled with adventitious regeneration utilizing nodal section explants and mature zygotic embryos. U.S. Pat. No. 6,570,068.
Xie, D. and Hong, Y. (2005) Regeneration and genetic transformation of Acacia mangium. U.S. Pat. No. 6,846,971.
Zhong, H., Boudreau, E., Rouse, S., Dunder, E., Gu, W. and Chang, Yin-Fu (2005) Methods for stable transformation of plants. U.S. Pat. No. 6,858,777.

The disclosures of the cited patent documents, publications and references are incorporated herein in their entirety to the extent not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A method of producing a transformed grapevine cell, the method comprising:
culturing a grapevine plant explant in culture medium comprising a growth regulator;
producing a plant shoot having a shoot meristem from said grapevine plant explant;
obtaining said shoot meristem from said grapevine plant explant;
exposing said shoot meristem to Agrobacteria comprising a vector; said vector comprising, operatively linked in the 5′ to 3′ orientation, a promoter that directs transcription of an exogenous structural nucleic acid sequence, an exogenous structural nucleic acid sequence, and a 3′ transcription terminator, thereby producing at least one transformed grapevine explant that comprises at least one cell having said exogenous structural nucleic acid sequence introduced therein; and
culturing said transformed grapevine explant in a selection medium.

2. The method of claim 1, wherein said method comprises wounding of said shoot meristem prior to said exposing step.

3. The method of claim 2, wherein said wounding comprises nicking or fragmenting said shoot meristem.

4. The method of claim 1, wherein said exposing comprises contacting and co-culturing said shoot meristem with said Agrobacteria.

5. The method of claim 1, wherein said shoot meristem comprises apical meristem tissue.

6. The method of claim 1, wherein said growth regulator is kinetin, benzylaminopurine, zeatin 2-ip, or kinetin, or a combination thereof.

7. The method of claim 1, wherein said vector also comprises a nucleic sequence encoding a selectable marker.

8. The method of claim 1, further comprising regenerating a plant from said transformed grapevine explant.

9. The method of claim 8, wherein said regenerating comprises culturing said transformed grapevine explant to form a transgenic shoot; culturing the
transgenic shoot to form a transgenic rooted shoot; and growing the transgenic rooted shoot to form a transgenic grapevine plant capable of expressing an exogenous structural nucleic acid sequence.

10. A method of producing a transformed grapevine plant, comprising:
culturing a grapevine plant explant in culture medium comprising a growth regulator;
producing a plant shoot having a shoot meristem from said grapevine plant explant;
introducing a nucleic acid into a cell of said shoot meristem using microprojectile bombardment, thereby producing a transformed grapevine cell comprising said nucleic acid; and
regenerating a transformed grapevine plant from said transformed grapevine cell.

11. The method of claim 10, wherein said shoot meristem is obtained from apical and/or axillary shoot meristem regions of said plant shoot.

12. The method of claim 10, wherein said growth regulator is kinetin, benzylaminopurine, zeatin 2-ip, or kinetin, or a combination thereof.

13. The method of claim 10, wherein said regenerating comprises
culturing said transformed grapevine explant to form a transgenic shoot; culturing the transgenic shoot to form a transgenic rooted shoot; and growing the transgenic rooted shoot to form a transgenic grapevine plant capable of expressing said nucleic acid sequence.

14. A method of producing a transformed grapevine plant, comprising:
culturing a grapevine plant explant in culture medium comprising a growth regulator;
producing a plant shoot having a shoot meristem from said grapevine plant explant;
introducing a nucleic acid into a cell of said shoot meristem using Agrobacteria comprising a vector; said vector comprising, operatively linked in the 5′ to 3′ orientation, a promoter that directs transcription of an exogenous structural nucleic acid sequence, an exogenous structural nucleic acid sequence, and a 3′ transcription terminator, thereby producing a transformed grapevine cell comprising said exogenous nucleic acid; and
regenerating a transformed grapevine plant from said transformed grapevine cell.

15. The method of claim 1, wherein said vector also comprises a nucleic sequence encoding a selectable marker.

16. The method of claim 14, wherein said growth regulator is kinetin, benzylaminopurine, zeatin 2-ip, or kinetin, or a combination thereof.

17. The method of claim 14, wherein said vector also comprises a nucleic sequence encoding a selectable marker.

18. The method of claim 14, wherein said regenerating comprises
culturing said transformed grapevine explant to form a transgenic shoot; culturing the transgenic shoot to form a transgenic rooted shoot; and growing the transgenic rooted shoot to form a transgenic grapevine plant capable of expressing said exogenous structural nucleic acid sequence.

19. The method of claim 14, wherein said shoot meristem is obtained from apical and/or axillary shoot meristem regions of said plant shoot.

20. A transformed grapevine plant explant produced during the method of claim 1.

21. The method of claim 1, wherein said growth regulator is an auxin.

22. The method of claim 1, wherein said growth regulator is a gibberellin.
